(19) United States
(12) Patent Application Publication
(10) Pub. No.: US 2021/0398659 A1
(43) Pub. Date: Dec. 23, 2021

(54) METHODS AND SYSTEMS FOR CONTACT TRACING OF OCCUPANTS OF A FACILITY

(71) Applicant: HONEYWELL INTERNATIONAL INC., Charlotte, NC (US)

(72) Inventors: Rajeev Sharma, Charlotte, NC (US); Amit Grewal, Charlotte, NC (US); Lalitha M Eswara, Charlotte, NC (US); Dinesh Babu Rajamanickam, Charlotte, NC (US); Sunil Madhusuthanan, Charlotte, NC (US)

(21) Appl. No.: 17/304,217

(22) Filed: Jun. 16, 2021

(30) Foreign Application Priority Data
Jun. 22, 2020 (IN) 202011026341

(51) Int. Cl. G16H 40/20

(52) U.S. Cl. CPC G16H 40/20

(57) ABSTRACT

A contact tracing system and method identifies which persons at a facility may have been exposed to a possibly contagious person. In some examples, multiple sensors, such as access card readers and cameras with video and thermographic imaging, monitor individuals at the facility. For each person, the system collects data pertaining to an individual's location and/or temperature at discrete points in time. In addition to collecting data throughout the day, data is also collected over an extended period of days or weeks. Based on data recorded during the extended periods, the system determines each person's typical schedule and travel pattern through the facility. When one or more sensors detect a possibly contagious person by sensing a fever, or even a gradually rising body temperature, the system searches for person's whose typical schedule and travel patterns overlap with those of the contagious person. The system then automatically notifies people accordingly.

### CROSS-REFERENCE TO RELATED APPLICATIONS

This application claims priority pursuant to 35 U.S.C. 119(a) of India Patent Application No. 202011026341, filed Jun. 22, 2020, which application is incorporated herein by reference in its entirety.

### TECHNICAL FIELD

The disclosure relates generally to contact tracing of individuals, and more particularly to methods and systems for contact tracing of individuals that may have been exposed to infection in a facility.

### BACKGROUND

Contact tracing is the process of identifying individuals that may have had firsthand exposure to a person with a contagious infection and/or secondhand exposure to another person who had firsthand exposure. Some methods of contact tracing branch out to extend beyond first and secondhand levels of exposure.

Contact tracing can be done manually, automatically or some combinations of the two. Manual methods tend to be labor intensive, time consuming and error prone. Automated methods often use cellphones or other devices that include integral GPS, Bluetooth proximity sensing and/or other location services to identify when two or more of the cellphones, and thus the people carrying the cell phones, have come near each other another.

### SUMMARY

The present disclosure generally pertains to a contact tracing system and method for identifying which persons at a facility may have been exposed to a possibly contagious person. In some examples, multiple sensors, such as access card readers and cameras with video and thermographic imaging, monitor individuals at the facility. For each person, the system collects data pertaining to an individual's location (and in some cases temperature) at discrete points in time. In addition to collecting data throughout the day, data is also collected over an extended period of days or weeks. Based on data recorded during the extended periods, the system determines each person's typical schedule and travel pattern through the facility. When a possibly contagious person is identified, the system searches for person's whose typical schedule and travel patterns overlap with the typical schedule of the contagious person.

In some examples of the disclosure, a method for identifying which if any of a plurality of people may have been exposed to an identified person in a facility includes storing location data of each of the of people in the facility over a period of time. The location data is synthesized to determine an typical occupancy pattern for each of the people. Each occupancy pattern identifies areas in the facility that were visited by the corresponding person over the period of time. Of the plurality of people, a particular person is identified. The occupancy pattern of the identified person is spatially compared with the occupancy pattern of each of the other people. The method includes identifying those other people that have an occupancy pattern with at least a threshold spatial overlap with the occupancy pattern of the identified person. A notification is provided identifying those other people that have an occupancy pattern with at least a threshold spatial overlap with the occupancy pattern of the identified person.

In some examples of the disclosure, a method for identifying occupancy patterns for each of a plurality of people in a facility includes storing location data of each of a plurality of people in the facility over a period of time. The location data is synthesized to determine an occupancy pattern for each of the plurality of people. Each occupancy pattern identifies areas in the facility that were visited by the corresponding one of the people over the period of time and areas within the facility where the corresponding person likely visited but such visit is not expressly represented in the location data. In some cases, the period of time includes two or more days.

In some examples of the disclosure, a non-transient computer readable medium storing instructions that when executed by a processor cause the processor to store location data of each of a plurality of people in a facility over a period of time. In some cases, the period of time is more than two days. The processor synthesizes the location data to determine a typical occupancy pattern for each of the plurality of people. Each occupancy pattern identifies areas in the facility that were visited by the corresponding one of the plurality of people over the period of time. The processor receives an identity of a particular person of the plurality of people. The processor spatially compares the typical occupancy pattern of the identified person with the typical occupancy pattern of each of the other plurality of people. The processor identifies those other people that have a typical occupancy pattern with at least a threshold spatial overlap with the typical occupancy pattern of the identified person. In some cases, the processor provides a notification identifying those other people that have a typical occupancy pattern with at least a threshold spatial overlap with the typical occupancy pattern of the identified person.

The preceding summary is provided to facilitate an understanding of some of the features of the present disclosure and is not intended to be a full description. A full appreciation of the disclosure can be gained by taking the entire specification, claims, drawings and abstract as a whole.

### BRIEF DESCRIPTION OF THE DRAWINGS

The disclosure may be more completely understood in consideration of the following description of various illustrative embodiments of the disclosure in connection with the accompanying drawings in which:

FIG. 1 is a schematic diagram of an example contact tracing system;

FIG. 2 is an example display screenshot of the computer system shown in FIG. 1;

FIG. 3 is a diagram illustrating an example database of the computer system shown in FIG. 1;

FIG. 4 is another diagram illustrating the example database of the computer system shown in FIG. 1;

FIG. 5 is a flow diagram showing an example method associated with the contact tracing system shown in FIG. 1;

FIG. 6 is a flow diagram showing another example method associated with the contact tracing system shown in FIG. 1;

FIG. 7 is a flow diagram showing another example method associated with the contact tracing system shown in FIG. 1;

FIG. 8 is a flow diagram showing another example method associated with the contact tracing system shown in FIG. 1; and,

FIG. 9 is a flow diagram showing another example method associated with the contact tracing system shown in FIG. 1.

While the disclosure is amendable to various modifications and alternative forms, specifics thereof have been shown by way of example in the drawings and will be described in detail. It should be understood, however, that the intention is not to limit the disclosure to the particular illustrative embodiments described herein. On the contrary, the intention is to cover all modifications, equivalents, and alternatives falling within the spirit and scope of the disclosure.

### DESCRIPTION

The following description should be read with reference to the drawings wherein like reference numerals indicate like elements throughout the several views. The description and drawings show several examples that are meant to be illustrative of the disclosure.

In some examples, the disclosure pertains to systems and methods for contact tracing of individuals that may have a contagious disease or may have been exposed to someone with an infection. Some of the examples are particularly suited for monitoring facilities where certain individuals work, attend or visit regularly. Examples of such facilities include places of employment, schools and hospitals. The currently disclosed systems and methods, however, can also be used in other settings, such as airports, cruise ships, and amusement parks.

In some examples, access card readers, surveillance cameras, infrared sensors, and/or other types of sensors record location and identity of individuals at various points of time as they travel within the facility. The points serve as “dots,” which can be connected to create a line that most likely represents each individual's travel route. The travel routes of one or more individuals of interest can be plotted on a map of the facility. The travel routes of one or more individuals of interest can be readily compared visually on the map and/or computer analyzed.

In some examples, the computer synthesizes collected data points over multiple days to identify an individual's most likely pattern of occupancy or travel route(s). Identifying such a pattern may be better defined and more useful than a relatively sparse one created based on data captured on a single day. Data collected on a single day, for example, may miss detecting an individual at certain key location points. For instance, on some days, an individual might forego scanning their access card, as the individual may have closely followed another person through a controlled access door. In some examples, a camera with facial recognition analytics might fail to detect or recognize an individual that may have had their face turned away or obstructed from view as they walked past the camera. Consequently, the computer can synthesize a larger sampling of data to help fill in these missing data points, and thereby create a predictive travel or occupancy model for each individual.

In some examples, the computer further analyzes data collected from one or more infra red sensors. The infrared sensors may be configured to measure a person's temperature at one or more times throughout the day and/or over multiple days. Some example infra red sensors are incorporated with a combination surveillance camera and infra red sensor. Other examples are standalone sensors independent of any surveillance camera.

In response to feedback from one or more infra red sensors, the computer identifies suspect individuals whose temperature may be increasing gradually or may have already risen to a level indicative of a fever. Such suspect individuals may then be tagged and traced. The suspect individual may be notified and/or restricted from accessing one or more areas of the facility. Others likely to have had contact with the suspect individual, even before detection of the fever, can also be notified and/or have their facility access restricted.

Some example systems and methods provide contact tracing at selective levels of time resolution based on the degree of detected activity or movement. If a relatively short time span (e.g., two hours) shows little or no movement, the search for matching occupancy and/or travel patterns can be applied over a longer time span (e.g., six hours). In some examples, a match is based on whether or not the same space is part of both typical occupancy patterns, and in some cases, likely occupied at or near the same time based on overlapping typical occupancy schedules for the space.

Some example systems and methods provide extended branch searching to identify not only individuals directly exposed to an infected person but to further identify additional individuals that may have had secondhand contact, i.e., contact with individuals who had direct contact with the infected person.

In some examples, the computer provides a map of the facility with various features and tools for user convenience. Some examples features and tools include being able to select and display the travel routes of various individuals, icons of cameras with video and/or image links, and links leading to meta data associated with chosen individuals, cameras, or access card readers.

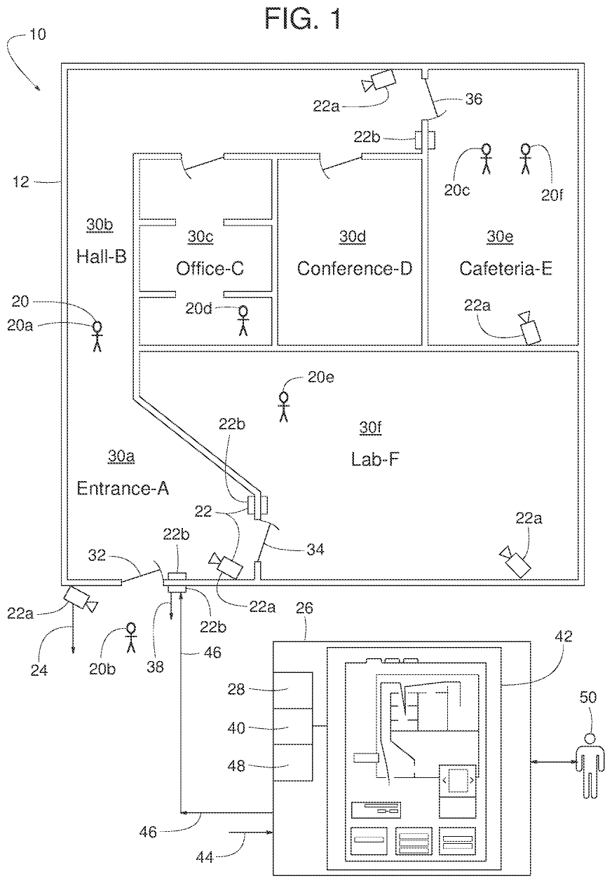

FIG. 1 is a schematic diagram illustrating one example of a contact tracing system 10 for use at a facility 12 (e.g., a single building, a group of buildings, an office, a hospital, a cruise ship, a hotel, an airport, a campus, a predefined outdoor area, a park, fairgrounds, a worksite, etc.). Contact tracing system 10 provides a method for identifying which, if any, of a plurality of people 20 (e.g., persons 20a-f) may have had direct or secondhand contact with a particular person 20 (e.g., person 20b) at facility 12. For sake of example, person 20b will be identified as possibly having a contagious infection (e.g., a seasonal flu, virus, bacteria, COVID-19, etc.).

In some examples, facility 12 includes a plurality of areas 30 such as rooms, zones, regions and/or hallways. For instance, in the illustrated example, facility 12 includes an Entrance-A 30a, a Hall-B 30b, an Office-C 30c (e.g., a plurality of work cubicles), a Conference-D 30d (conference room), a Cafeteria-E 30e, and a Lab-F 30f.

A plurality of sensors 22 are installed for monitoring the travel routes or occupancy patterns of people 20 in and around facility 12. Sensors 22 are schematically illustrated to represent any type of device capable of detecting, recognizing and/or identifying persons 20. Some examples of sensors 22 include cameras, video cameras, surveillance cameras, CCTV cameras, still picture cameras, infrared sensors, access card readers, electric eyes, microphones, motion sensors, etc., and various combinations thereof.

Some examples of sensor 22 include a camera 22a in a single housing that contains both a visible light video module and a thermographic module. In other examples, the modules are in separate housings. The visible light video module captures pictures or videos. The thermographic module measures the body temperature of a person. It should be noted, however, that sensor 22a is schematically illustrated to represent any thermal camera capable of detecting a body temperature in fever range.

In some examples, camera 22a provides an output 24, which is conveyed to a computer system 26 for storage in a database 28 and later retrieved for contact tracing analysis. Some examples of output 24 include video clips, video images, motion detection signal, meta data, body temperature readings, a recording timestamp, an alarm signal, camera location, camera serial number, and various combinations thereof. In some examples, computer system 26 applies video analytics (e.g., face recognition software) to identify each person 20 in the captured videos and images.

In addition or alternatively, some example sensors 22 of contact tracing system 10 include an access card reader 22b (access control device) at doors 32, 34 and 36. In some examples, access card readers 22b detect identification cards of persons 20 as their card is swiped, entered or placed in sensing proximity with access card reader 22b . In response to detecting a person's identification card, access reader 22b provides an output 38. Examples of output 38 include meta data or information pertaining to person 20 (e.g., person's name or employee identification number); information of the access reader itself (e.g., reader location, door location; reader serial number, etc.); a timestamp of card detection; a signal indicating whether the person's card was accepted or rejected; a signal to lock or unlock door 32, 34 or 36; an alarm signal; and/or various combinations thereof. In some examples, output 38 is conveyed to computer system 26 for storage in database 28 and later retrieved for contact tracing analysis.

In some examples, computer system 26 includes a processor 40, database 28 for storing data, a display 42 for providing a user interface, a plurality of inputs 44, a plurality of outputs 46, and a non-transitory computer readable medium 48. Non-transitory computer readable medium 48 contains instructions or program codes that when executed by computer system 26 causes computer system 26 to perform one or more of the method steps disclosed herein. The term, “computer readable medium” refers to any device for storing information for any duration (e.g., for extended time periods, permanently, for brief instances, for temporarily buffering, for caching of the information, etc). The term, “program code” refers to executable instructions (e.g., computer readable instruction, machine readable instructions, software, etc.). The term, “non-transitory computer readable medium” is specifically defined to include any type of computer readable storage device and/or storage disk and to exclude propagating signals and to exclude transmission media. In some examples, components of computer system 26 are contained in one location. In other examples, components of computer system 26 are physically spaced apart but interconnected in signal communication with each other.

Some examples of the computer system's inputs 44 include output 38 from card reader 22b, output 24 from camera 22a, keyboard signals from a user 50, mouse- click signals from user 50, and touchscreen inputs from user 50. In response to inputs 44, computer system 26 provides outputs 46. Examples of outputs 46 include an email notification or alert, a SMS text message or alert, a notification to one or more persons 20 via the person's mobile device (e.g., cellphone), a map and/or instructions to a cleaning crew to decontaminate exposed regions of facility 12, and/or signals to access readers 22b that prevent certain people from passing through certain doors.

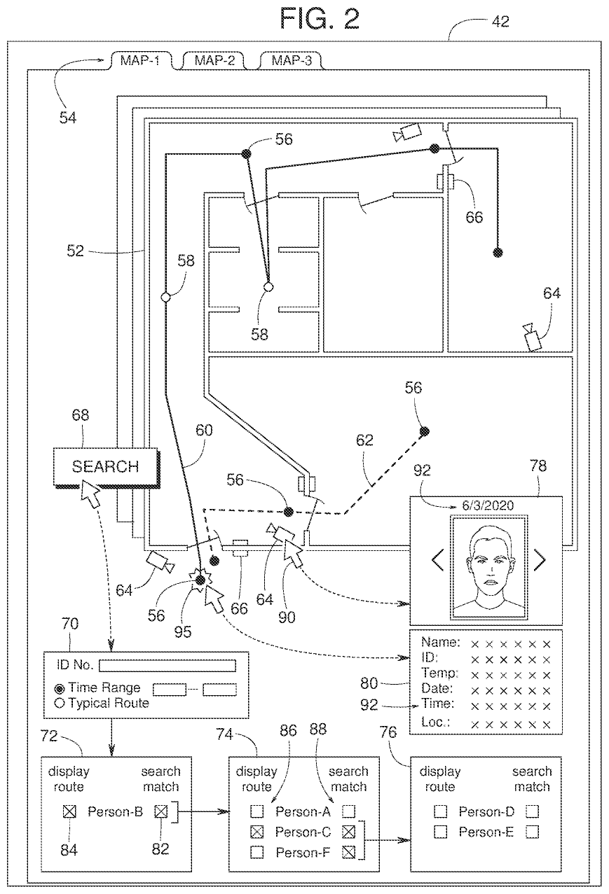

In some examples, as shown in FIG. 2, display 42 of computer system 26 displays various screens that provide user 50 with convenient features, links, and useful information for contact tracing of people 20. In the illustrated example, display 42 shows a map 52 (e.g., 2D map, 3D model, etc.) of at least one area or floor of facility 12, tabs 54 for selecting maps 52 of different areas or floors of facility 12, solid points 56 on map 52 indicating where sensors 22 actually detected one or more persons 20 at certain locations in facility 12, and hollow points 58 indicating where one or more persons 20 may have likely been based on detections at nearby locations. In some examples, hollow points 58 indicate where a certain person 20 may have likely visited but such visit is not expressly represented in the location data. For example, since person 20b was detected by the entrance at solid dot 56 and also outside Office-C 30c, the person 20b must have passes through Hall-B 30b, as indicated by hollow point 58 in Hall-B 30b.

In some examples, display 42 may also show a line 60 representing a likely travel route of a chosen person (e.g., person 20b), another line 62 representing a likely travel route of another person (e.g., person 20e), camera icons 64 showing where cameras 22a are located, reader icons 66 showing where access readers 22b are located, a search button 68 for initiating a search for a particular person (e.g., person 20b) and/or for other persons 20 having direct or secondhand exposure to the particular person, and a plurality of popup windows 70, 72, 74, 76, 78 and 80.

In some examples, window 70 enables user 50 to enter a chosen person (e.g., person 20b) for which to search. The person can be identified by name, employee number, or by some other means for identification (e.g. picture of the person's face). Window 70 also enables user 50 to specify a time range over which to search for sightings or detections of the chosen person and/or to specify that the chosen person's typical travel route (and/or typical occupancy pattern) be displayed on map 52. The usual travel route (and/or typical occupancy pattern) is derived by synthesizing a person's location data collected over an extended period of perhaps days or even weeks.

Popup window 70 leads to popup window 72. Popup window 72 identifies the particular person (e.g., person 20b) for which contact tracing system 10 is to find other people 20 that may have had contact with person 20b . In some examples, user 50 selects a checkbox 82 to initiate the search to find those that may have had contact with person 20b . To do this, computer system 26 accesses database 28 to find which of people 20 have a typical occupancy pattern with at least a threshold spatial overlap with the typical occupancy pattern of person 20b.

The term, “spatial overlap” in the comparison of two people means that the two likely occupied a common space sometime during a certain duration of time (e.g. last two weeks). The term, “threshold spatial overlap,” is what defines the size or span of a certain physical range or location, and further defines the length of the certain duration of time. In some examples, the size or span of the certain physical range or location might be specified as two people being likely occupying the same room (e.g., Office-C 30c, Cafeteria-E 30e, etc.), while the length of the certain duration of time may be a defined as anything greater than zero seconds or greater than a few minutes to days or even weeks.

In some examples, popup window 72 includes another checkbox 84. Checkbox 84 enables user 50 to specify that a typical travel route of the particular person (e.g., person 20b) be displayed on map 52.

In some examples, contact tracing system 10 displays the results of a direct contact tracing search in popup window 74. Popup window 74 lists one or more persons (e.g., persons 20a, 20c and 20f) that have had contact (e.g., above a threshold spatial overlap) with person 20b . Checkboxes 86 enable user 50 to specify which, if any, typical travel routes of persons 20a, 20c and/or 20f should be displayed on map 52. In some examples, popup window 74 includes additional checkboxes 88 for initiating extended secondhand contact tracing. Secondhand contact tracing identifies persons (e.g., persons 20d and 20e) that have contact (e.g., above a threshold spatial overlap) with the selected persons (e.g., persons 20c and 20f) checked in popup window 74. In this example, persons 20d and 20e are displayed in popup window 76, which indicates they have shared spatial overlap with at least one of persons 20c or 20f.

In some examples, mouse-clicking 90 or otherwise selecting a camera icon 64 links to popup window 78, which shows photos and/or videos captured by corresponding cameras 22a . in some examples, popup window 80 displays tagged metadata associated with the photos or videos and/or associated with a particular solid dot 56. Some example metadata include a timestamp 92 and/or location indicating when and/or where the photo or video was taken.

In some examples, mouse-clicking or otherwise selecting a particular dot icon 56 links to popup window 80, which shows various information or metadata pertaining to dot icon 56. Some example information includes the name of the relevant person 20 corresponding to that dot icon, person identification number, body temperature of the relevant person 20, date and time of detection by the sensor 22 (e.g., camera 22a). In some examples, a dot icon 56 encircled by a star 95 or otherwise highlighted indicates a detected person 20 with a fever.

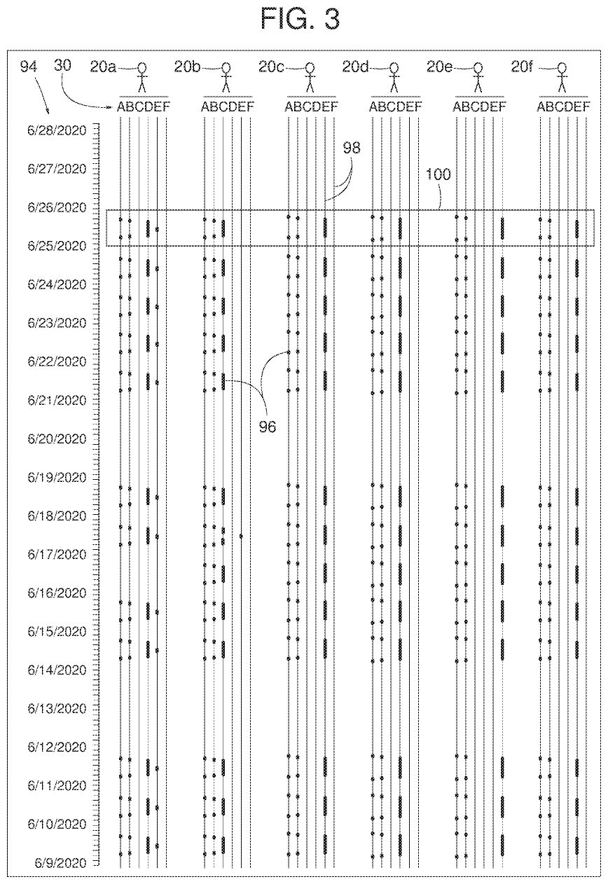

In some examples, the structure of database 28 can be visualized with the diagram shown in FIGS. 3. In this diagram, persons 20a-f are shown along the top horizontal row. Under each person 20 there is a short horizontal segment showing each of the six areas 30 (Entrance-A 30a, Hall-B 30b, Office-C 30c, Conference-D 30d, Cafeteria-E 30e, and Lab-F 30f) represented by respective letters A-F. For example, letter “A” under each person 20 corresponds to Entrance-A 30a, letter “B” under each person 20 corresponds to Hall-B 30b, etc. The left column shows a vertical time scale 94 of, for example, days, hours, minutes, and/or seconds. So, the left column represents a dimension of time, while the upper column represents a dimension of space or location.

Relatively thick lines 96 on relatively thin vertical guidelines 98 represent when a particular person 20 is at a particular location at a particular time. Some lines 96 are so vertically short they appear to be dots. Such “dots” can be the result of a person 20 spending, for example, just a few seconds at an access reader 22, while a longer line 96 represents a person 20 staying at one location (e.g., at Office-C 30c) for an extended period.

In viewing FIG. 3, typical occupancy patterns of persons 20 emerge. For example, person 20d regularly spends time in Conference-D 30d; person 20e spends most of their time in Lab-F 30f; persons 20b, 20d, and 20e never visit Cafeteria-E 30e; and nobody works on the weekends.

A few irregularities also appear. Person 20a, for example, was absent for one day on June 16. And on June 17, person 20b chose to eat lunch in Cafeteria-E 30e instead of in the usual place of Office-C 30c.

Despite the irregularities, relatively consistent occupancy patterns may emerge for each person 20. So, in some examples, computer system 26 applies predictive analytics, which may include a process of synthesizing each person's location data over a period of time (e.g., over two or more days or even weeks), as shown in FIG. 3, and thereby determining each person's normal, expected or typical occupancy pattern to reflect an aggregate representation of the areas visited by each person 20. In some examples, occupancy patterns based on synthesizing location data reveals locations a particular person 20 likely visited but is not expressly represented in the location data.

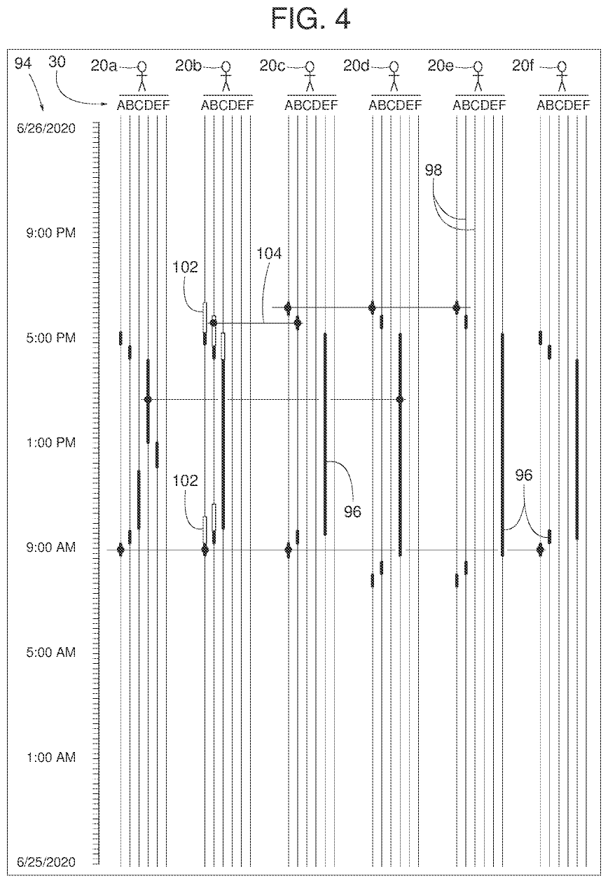

In some case, rather than using “typical” occupancy patterns, a more granular time and space comparison may be made. FIG. 4 is an enlarged view FIG. 3 but covering just a one-day time period 100 on June 25. FIG. 3 shows examples of spatial overlap of people 20 at certain times and locations. With reference to the search example shown in FIG. 2, person 20b has spatial overlap with persons 20a, 20c and 20f at Entrance-A 30a at 9:00 AM. An extended search for secondhand contact shows person 20a having spatial overlap with person 20d in Conference-D 30d at around 2:00 PM. And person 20c is shown having spatial overlap with persons 20d and 20e at Entrance-A 30a at around 6:00 PM. Thus computer systems 26 may identify person 20b having contact (with at least a predefined threshold spatial overlap) with persons 20a, 20c and 20f An extended search finds that persons 20c and/or 20f had contact (with at least a predefined threshold spatial overlap) with persons 20d and 20e.

In some examples, the level of exposure is ranked with an exposure score based on the duration or dwell time (or an aggregation of time) in which two or more people were in the same general location. In some examples, the dwell time needs to exceed a predetermined threshold amount of time. In some examples, the rank of secondhand level of exposure is limited to no more than the prior direct exposure event from which the secondhand exposure stems. For instance, in an example where person 20a had ten minutes of firsthand exposure to an infected person 20b, and person 20d had three hours of secondhand exposure to person 20a, then the rank of person 20a would be based on ten minutes, and the rank of person 20d would be limited to no more than ten minutes even though person 20d had three hours of contact with person 20a.

In some examples, computer system 26 adjusts a person's occupancy pattern to account for situations where an exposed person might not have shared the same space and time as an infected person, but the exposed person may have entered a space shortly after the infected person left that space. To account for this, some examples of computer system 26 add a virtual “cloud of dust” that follows and lingers in the path of an infected person. Such a “cloud of dust” is visually depicted by adding vertical extensions 102 above the upper ends of thick lines 96. In the example of infected person 20b, shown in FIG. 4, extensions 102 and line 104 show that persons 20b and 20c experienced spatial overlap in Hall-B 30b, as person 20c was in Hall-B 30b shortly after person 20b was in Hall-B 30b . In some examples, the time length of extension 102 is user adjustable.

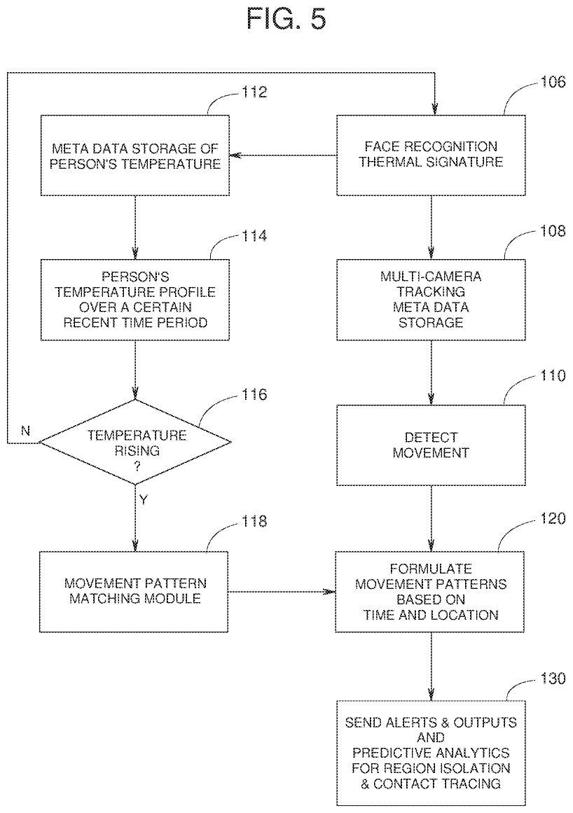

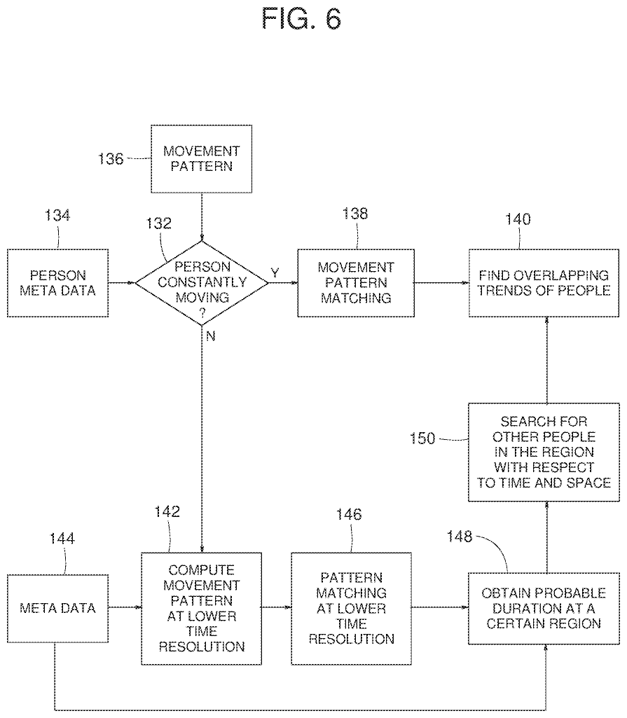

In some examples, contact tracing system 10 and its associated methods are structured as shown in FIGS. 5 and 6. A block 106 represents camera 22a capturing images, video and body temperatures of people 20. In some examples, video analytics are applied to identify individuals using known facial recognition software. A block 108 represents multiple cameras 22a collecting images with metadata at multiple locations capturing and storing both the images and their associated metadata on database 28 of computer system 26. A block 110 represents sensor 22 detecting movement of persons 20. A block 112 represents the sensing and recording the body temperature of individuals 20 and doing so repeatedly over a period of time. The recorded body temperatures are stored as metadata in database 28. A block 114 represents identifying a person's temperature profile as it may vary over time, wherein the body temperature profile is based on the recorded body temperatures stored in database 28. A block 116 represents determining whether or not a person's body temperature profile indicates a rising temperature. If a particular person's body temperature is rising, and perhaps exceeding a predetermined upper limit, the contact tracing process proceeds to a movement pattern matching module 118, which is outlined in FIG. 6. Module 118 leads to a block 120 in FIG. 5. Block 120 represents formulating the particular person's travel and occupancy patterns based on detecting the person's various locations over time. A person's location may be the person's actual detected location, an undetected location based on an interpolation of nearby actual detections, and/or a predicted location based on an observed pattern of the person's usual travel routes. A block 130 represents sending appropriate alerts (e.g., email notifications, SMS text messages to mobile devices), and/or outputs (e.g., signals to access readers 22b for preventing certain people from passing through certain doors).

Movement pattern matching module 118, of FIG. 5, may function as shown in FIG. 6. A block 132 of FIG. 6 represents determining whether a person's travel pattern (occupancy pattern) indicates at least a certain amount of travel movement over a predefined period of time (e.g., two hours). Blocks 134 and 136 provides block 132 with metadata identifying a particular person 20 and their travel pattern. If the person's travel pattern includes sufficient movement, block 138 compares the travel patterns of others. A block 140 represents identifying travel patterns with at least a threshold spatial overlap.

If block 132 determines that a particular person's travel movement is insufficient over the predetermined period of time (e.g., two hours), then block 142 computes the particular person's travel pattern over a longer period (e.g., six hours) and does so based on metadata from block 144. While using the longer timeframe, block 146 represents comparing the travel patterns of others to help identify travel patterns with at least a threshold spatial overlap. A block 148, in some examples, represents determining a person's probable dwell or duration at certain areas of facility 12 and does so based on metadata from block 144. A block 150 represents searching in those areas for others having an overlap with respect to time and location.

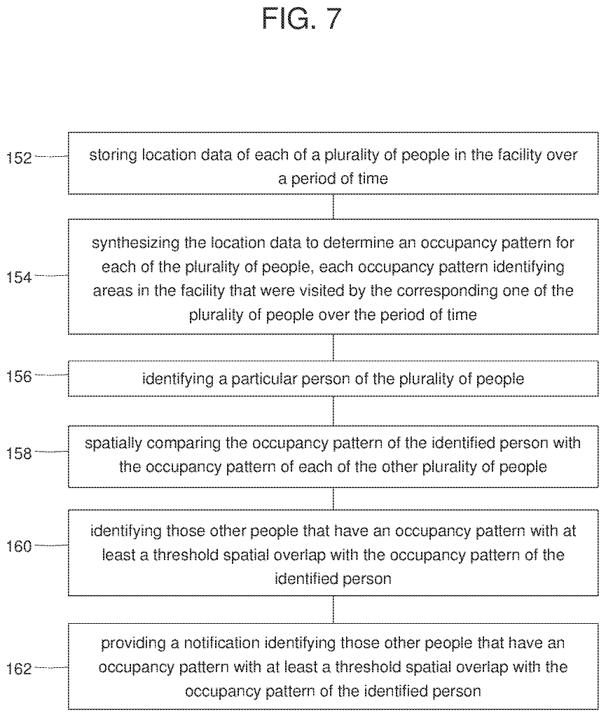

FIG. 7 is a flow diagram illustrating some example methods steps of an example method for identifying which, if any, of a plurality of people may have been exposed to an identified person in a facility. In some examples, blocks 152, 154, 156, 158, 160, and 162 may be performed by at least one of computer system 26, its processor 40, computer readable medium 48, display 42, inputs 44, outputs 46, database 28, and sensors 22.

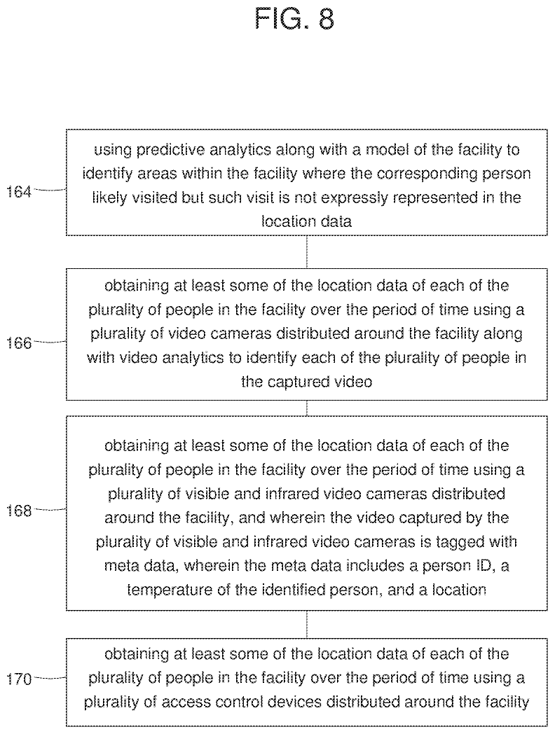

FIG. 8 is a flow diagram illustrating some additional example methods steps of the example method for identifying which, if any, of a plurality of people may have been exposed to an identified person in a facility. In some examples, blocks 164, 166, 168, and 170 may be performed by at least one of computer system 26, its processor 40, computer readable medium 48, display 42, inputs 44, outputs 46, database 28, and sensors 22..

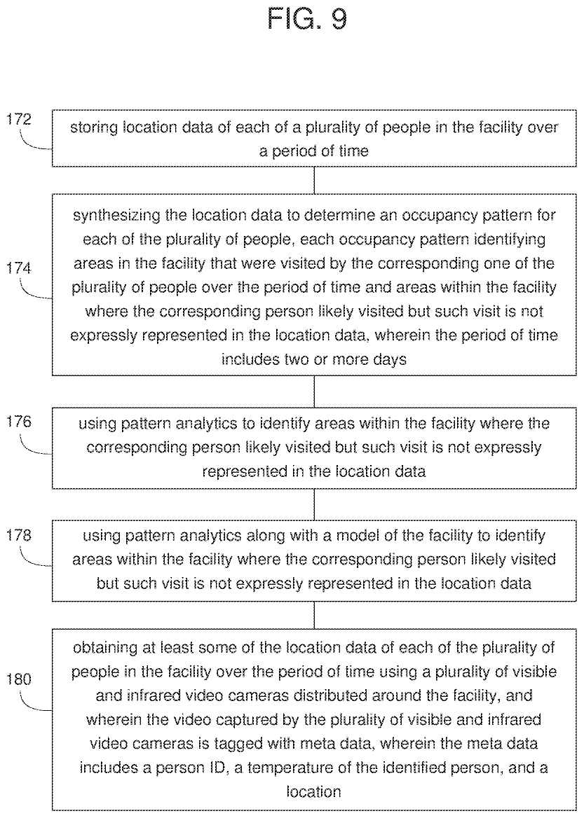

FIG. 9 is a flow diagram illustrating some example methods steps for identifying occupancy patterns for each of a plurality of people in a facility. In some examples, blocks 172, 174, 176, 178, and 180 may be performed by at least one of computer system 26, its processor 40, computer readable medium 48, display 42, inputs 44, outputs 46, database 28, and sensors 22.

The disclosure should not be considered limited to the particular examples described above. Various modifications, equivalent processes, as well as numerous structures to which the disclosure can be applicable will be readily apparent to those of skill in the art upon review of the instant specification.

What is claimed is:

1. A method for identifying which if any of a plurality of people may have been exposed to an identified person in a facility, the method comprising:
storing location data of each of the plurality of people in the facility over a period of time;
synthesizing the location data to determine an occupancy pattern for each of the plurality of people, each occupancy pattern identifying areas in the facility that were visited by the corresponding one of the plurality of people over the period of time;
identifying a particular person of the plurality of people;
spatially comparing the occupancy pattern of the identified person with the occupancy pattern of each of the other plurality of people;
identifying those other people that have an occupancy pattern with at least a threshold spatial overlap with the occupancy pattern of the identified person; and
providing a notification identifying those other people that have an occupancy pattern with at least a threshold spatial overlap with the occupancy pattern of the identified person.

2. The method of claim 1, wherein the period of time is more than two days.

3. The method of claim 1, wherein the areas identified by the occupancy pattern include one or more of: rooms, zones, regions and/or hallways of the facility.

4. The method of claim 1, wherein each occupancy pattern further identifies a dwell time for each of the areas in the facility that were visited by the corresponding one of the plurality of people over the period of time.

5. The method of claim 4, wherein the dwell time represents an aggregation of the time that the corresponding one of the plurality of people spent in the corresponding area over the period of time.

6. The method of claim 1, wherein each occupancy pattern represents an aggregate representation of the areas in the facility that were visited by the corresponding one of the plurality of people over the period of time.

7. The method of claim 1, wherein each occupancy pattern identifies areas in the facility that were visited by the corresponding one of the plurality of people for more than a threshold amount of time.

8. The method of claim 1, wherein synthesizing the location data to determine the occupancy pattern for each of the plurality of people comprises using predictive analytics along with a model of the facility to identify areas within the facility where the corresponding person likely visited but such visit is not expressly represented in the location data.

9. The method of claim 1, further comprising:
obtaining at least some of the location data of each of the plurality of people in the facility over the period of time using a plurality of video cameras distributed around the facility along with video analytics to identify each of the plurality of people in the captured video.

10. The method of claim 1, further comprising:
obtaining at least some of the location data of each of the plurality of people in the facility over the period of time using a plurality of visible and infrared video cameras distributed around the facility, and wherein the video captured by the plurality of visible and infrared video cameras is tagged with meta data, wherein the meta data includes a person ID, a temperature of the identified person, and a location.

11. The method of claim 1, further comprising:
obtaining at least some of the location data of each of the plurality of people in the facility over the period of time using a plurality of access control devices distributed around the facility.

12. The method of claim 1, wherein the identified person is identified as having a contagious disease.

13. A method for identifying occupancy patterns for each of a plurality of people in a facility, the method comprising:
storing location data of each of a plurality of people in the facility over a period of time;
synthesizing the location data to determine an occupancy pattern for each of the plurality of people, each occupancy pattern identifying areas in the facility that were visited by the corresponding one of the plurality of people over the period of time and areas within the facility where the corresponding person likely visited but such visit is not expressly represented in the location data; and
wherein the period of time includes two or more days.

14. The method of claim 13, wherein synthesizing the location data comprises using pattern analytics to identify areas within the facility where the corresponding person likely visited but such visit is not expressly represented in the location data.

15. The method of claim 13, wherein synthesizing the location data comprises using pattern analytics along with a model of the facility to identify areas within the facility where the corresponding person likely visited but such visit is not expressly represented in the location data.

16. The method of claim 13, further comprising:
obtaining at least some of the location data of each of the plurality of people in the facility over the period of time using a plurality of visible and infrared video cameras distributed around the facility, and wherein the video captured by the plurality of visible and infrared video cameras is tagged with meta data, wherein the meta data includes a person ID, a temperature of the identified person, and a location.

17. A non-transient computer readable medium storing instructions that when executed by a processor cause the processor to:
store location data of each of a plurality of people in a facility over a period of time that is more than two days;
synthesize the location data to determine an occupancy pattern for each of the plurality of people, each occupancy pattern identifying areas in the facility that were visited by the corresponding one of the plurality of people over the period of time;
receive an identity of a particular person of the plurality of people;
spatially compare the occupancy pattern of the identified person with the occupancy pattern of each of the other plurality of people;
identify those other people that have an occupancy pattern with at least a threshold spatial overlap with the occupancy pattern of the identified person; and
provide a notification identifying those other people that have an occupancy pattern with at least a threshold spatial overlap with the occupancy pattern of the identified person.

18. The non-transient computer readable medium of claim 17, wherein the instructions cause the processor to:
identify areas within the facility where the corresponding person likely visited but such visit is not expressly represented in the location data.

19. The non-transient computer readable medium of claim 17, wherein the areas identified by the occupancy pattern include one or more of: rooms, zones, regions and/or hallways of the facility.

20. The non-transient computer readable medium of claim 17, wherein each occupancy pattern represents an aggregate representation of the areas in the facility that were visited by the corresponding one of the plurality of people over the period of time.
